(19) United States
(12) Patent Application Publication
(10) Pub. No.: US 2021/0007872 A1
(43) Pub. Date: Jan. 14, 2021

(54) Sports Tape for Joint PLacement

(71) Applicant: Taylor Shipley, Encino, CA (US)

(72) Inventor: Taylor Shipley, Encino, CA (US)

(21) Appl. No.: 16/510,769

(22) Filed: Jul. 12, 2019

(51) Int. Cl. A61F 5/01; B32B 5/02; B32B 7/12; B32B 38/00

(52) U.S. Cl. CPC A61F 5/0106; B32B 5/022; B32B 7/12; B32B 2405/00; B32B 38/0004; B32B 38/145; A61F 5/0118

(57) ABSTRACT

A sports tape for placement at a user's knee or elbow includes a first stabilizer portion and a second stabilizer portion configured for placement on opposite sides of the user's knee or elbow. A central portion comprising a bar or circle connects the first stabilizer portion and the second stabilizer portion and is configured to wrap around the user's knee or elbow. A first stabilizer is embedded in the first stabilizer portion, and a second stabilizer is embedded in the second stabilizer portion, disposed inside the sports tape, sandwiched between sports tape layers to provide strength and resiliency to the sports tape.

### BACKGROUND

### Field of the Invention

The present invention generally relates to athletic taping for sports activities. More specifically, the present invention relates to athletic tape for application to an athlete's joints, including knees, elbows, shoulders and fingers, among others.

### Related Art

Athletic tape is known in the art, and is typically applied directly to the skin in order to maintain a stable musculature at points of flexion, such as at knee and elbow joints, and including shoulders and fingers. Although a specialty apparatus may be procured with the aid of a physician, these are typically expensive and are not intended as a non-prescription solution to joint weakness and pain. Currently known in the art are four basic types of off-the-shelf apparatus uses by athletes with joint ailments. These include braces, compression sleeves, athletic tape, and kinesiology tape.

Non-prescription braces are typically made of polyester, spandex, or a blend of the two. They are usually bulky and incorporate mundane designs and colors, intended for older consumers. Braces are disfavored because they must be periodically readjusted throughout the day or during a given activity, and require washing after use. Compression sleeves are similar in structure, but fail to function as a joint support. This is because compression sleeves offer almost no support, and function more as muscle warmers and/or fashion accessories. Athletic tape is arguably the best currently known solution for addressing joint ailments, but can be difficult to use. Proper taping techniques must be learned and studied, and the conventional athletic tape is very difficult to apply without a partner or trainer's assistance. Kinesiology tape is similar to athletic tape, but is an elastic adhesive tape, but has shown no effectiveness for joint ailments.

Hence, what is needed is an athletic tape that combines the support of a brace and the flexibility and convenience of a kinesiology tape. Additionally, an athletic tape is needed that can be easily applied and removed, for example, in a manner similar to a band aid. An athletic tape is also needed including stabilizers that help keep joints—including knee or elbow joints—aligned, which is secure during sports activities, and which can simply be peeled off and discarded after use. These, and other object of the present invention are more fully disclosed in the following summary, description, and claims.

### SUMMARY

A sports tape for placement at the location of a joint, including a user's knee, elbow, shoulder, or fingers, includes a first stabilizer portion and a second stabilizer portion, with the first stabilizer portion configured for placement on one side of the user's knee or elbow joint, and the second stabilizer configured for placement on an opposing side of the user's knee or elbow joint. A central portion connects the first stabilizer portion and the second stabilizer portion, with the central portion configured to wrap around the front of a user's knee or rear of the user's elbow joint. A first stabilizer is embedded in the first stabilizer portion, and a second stabilizer is embedded in the second stabilizer portion. The first stabilizer and the second stabilizer are preferably each centered on the first stabilizer portion and the second stabilizer portion, respectively. The first stabilizer and the second stabilizer are preferably disposed inside the sports tape, sandwiched between sports tape layers.

The sports tape preferably comprises a non-woven tape with adhesive, and the stabilizers comprise steel corset boning material. The sports tape also preferably comprises a transfer tape adhesive for adhering the sports tape to the user's knee, elbow, shoulder, or finger. The first stabilizer portion and the second stabilizer portion are also preferably elongated members with a middle bend in the direction of the user's joint, for example, knee or elbow joint. In some preferred embodiments, the central portion may be a bar between the first stabilizer portion and the second stabilizer portion. In other preferred embodiments, the central portion is a planar circle, with the first stabilizer portion and the second stabilizer portion located on opposing sides of the circle. Preferably, the sports tape also includes a non-woven tape layer covering the first stabilizer and a non-woven tape layer covering the second stabilizer.

The manufacture of the sports tape for placement at the location of a user's knee, elbow, shoulder, or finger joints, includes a method incorporating in general the following steps: A sheet of non-woven tape is obtained, the sheet of non-woven tape having an adhesive side and a non-adhesive side. A sheet of transfer tape adhesive is then obtained, along with a length of corset boning. Thereafter a design according to the desired shape of the sports tape is printed on the non-woven tape. Preferably a design is printed corresponding to the sports tape outline on the non-adhesive side of the non-woven tape. Thereafter, the transfer tape adhesive is laminated to the adhesive side of the non-woven tape.

The non-woven tape is cut, after laminating, into a plurality of sports tape shapes for placement at the location of a user's joint (i.e., knee, elbow, shoulder, or finger joint), and a plurality of second non-woven tape layers, and the boning is placed on the transfer tape adhesive and encased between the sports tape and one of the second non-woven tape layers. Preferably, this is accomplished by placing one of the second non-woven tape layers over the boning on the printed side of the second non-woven tape layer, thereby forming a uniform transfer tape adhesive coverage over the boning. In one exemplary embodiment, the sports tape is then placed proximal the user's knee joint for use. In other embodiments, the sports tape may be placed proximal the user's elbow, shoulder, or finger.

In some embodiments, the method may include the step of cutting the non-woven tape, after laminating, into a shape having a bar configured for placement at the user's knee, elbow, shoulder or finger joint. The non-woven tape may also be made, after laminating, into a shape having a planar circle configured for placement at the user's joint. Additionally, the non-woven tape may be cut, after laminating, to form two stabilizer portions on either side of a central portion. The stabilizer portions are preferably angled complimentary to the user's knee, elbow, shoulder, or finger joint, and the lengths of corset boning are located on the stabilizer portions, including being centered on the stabilizer portions. In another preferred embodiment, a wax paper layer is placed on the transfer tape adhesive opposite the non-woven tape.

After manufacture, one method of using the improved sports tape includes the steps of shaving the area around the user's knee, elbow, shoulder, or finger joint as necessary, applying rubbing alcohol around the area around the user's joint, and in the case of a knee or elbow joint, matching the user's knee or elbow joint to a pair of side stabilizers on the sports tape. The backing is peeled off the sports tape to expose the transfer tape adhesive, and while centering the sports tape, the middle of the sports tape is placed adjacent the middle of the user's knee or elbow joint. Working the sports tape against the user's knee or elbow from the center outward, the sports tape is held against the user's knee or elbow joint for approximately twenty seconds. The user may then engage in athletic activity while wearing the sports tape.

### BRIEF DESCRIPTION OF THE FIGURES

FIG. 1 illustrates a perspective view of a first embodiment sports tape for knee and elbow placement, having a central circle;

FIG. 2 illustrates a plan view of the first embodiment sports tape;

FIG. 3 illustrates a second embodiment sports tape for knee and elbow placement having a central bar;

FIG. 4 illustrates a plan view of the second embodiment sports tape;

FIG. 5 illustrates a section view of the sports tape for knee and elbow placement

FIG. 6A illustrates a plan view of a flat non-woven tape and transfer adhesive tape printed with the first embodiment sports tape for knee and elbow placement;

FIG. 6B illustrates a plan view of a flat non-woven tape and transfer adhesive tape printed with the second embodiment sports tape for knee and elbow placement;

FIG. 7 illustrates a plan view of a flat non-woven tape and transfer adhesive tape printed with an additional embodiment for fitting to a user's ankle;

FIG. 8 illustrates a plan view of a flat non-woven tape and transfer adhesive tape printed with an alternative additional embodiment for fitting to user's ankle;

FIG. 9 illustrates a plan view of a flat non-woven tape and transfer adhesive tape printed with another alternative additional embodiment for fitting to a user's ankle;

FIG. 10 illustrates a plan view of a flat non-woven tape and transfer adhesive tape printed with an additional embodiment for fitting to a user's shoulder;

FIG. 11 illustrates a plan view of a flat non-woven tape and transfer adhesive tape printed with an alternative additional embodiment for fitting to a user's shoulder;

FIG. 12 illustrates a plan view of a flat non-woven tape and transfer adhesive tape printed with another alternative additional embodiment for fitting to a user's shoulder; and

FIG. 13 illustrates a plan view of a flat non-woven tape and transfer adhesive tape printed with an additional embodiment for fitting to a user's finger.

### DESCRIPTION

The following description is presented to enable any person skilled in the art to make and use the invention, and is provided in the context of a particular application and its requirements. Various modifications to the disclosed embodiments will be readily apparent to those skilled in the art, and the general principles defined herein may be applied to other embodiments and applications without departing from the spirit and scope of the present invention. Thus, the present invention is not limited to the embodiments shown, but is to be accorded the widest scope consistent with the principles and features disclosed herein.

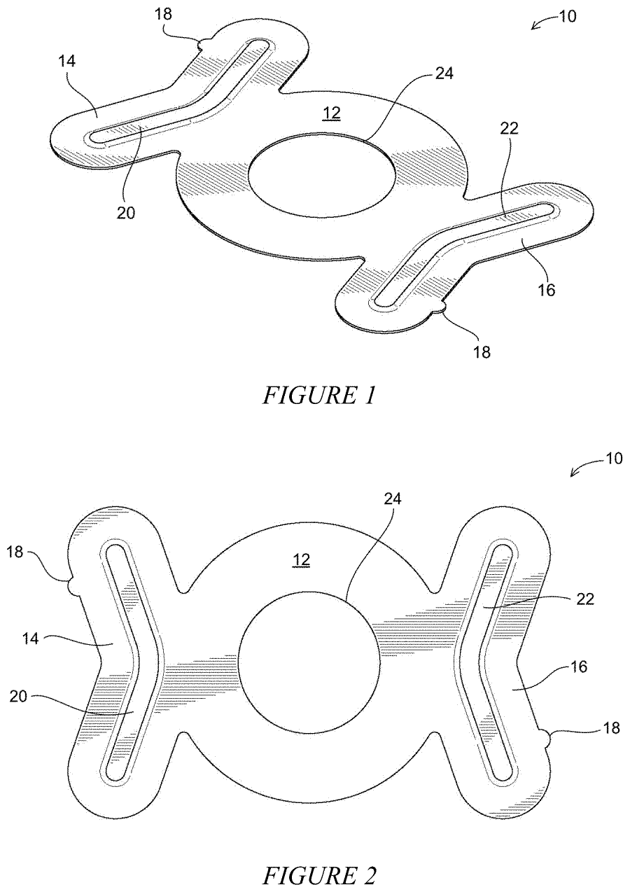

Referring to FIGS. 1 and 2, a first embodiment sports tape is shown for knee and elbow placement (sports tape) 10. In other embodiments, a substantially similar sports tape 10 may be shaped and/or sized for a user's shoulder or finger. The sports tape 10 includes a central circular portion 12 between a first stabilizer portion 14 and a second stabilizer portion 16. The first stabilizer portion 14 and the second stabilizer portion 16 are preferably arced or chevron-shaped members, with the concave portion of the first stabilizer portion 14 and the second stabilizer portion 16 aimed away from the circular portion 12. Each of the first stabilizer portion 14 and the second stabilizer portion 16 include a tab 18 for pulling the sports tape 10 apart, and in some instances exposing an adhesive backing and adhering the sports tape 10 to a user's knee or elbow.

Still referring to FIGS. 1 and 2, the first stabilizer portion 14 includes an embedded first stabilizer 20, and the second stabilizer portion 16 includes an embedded second stabilizer 22. The first stabilizer 20 and the second stabilizer 22 preferably follow the arc or chevron-shape of the first stabilizer portion 14 and second stabilizer portion 16, respectively, and in one embodiment are centered thereon as shown in the figures. The first stabilizer 20 and the second stabilizer 22 preferably comprise a resiliently deformable material. In one preferred embodiment, the first stabilizer 20 and the second stabilizer 22 are made from steel spiral corset boning, including safety caps (not shown) at their ends to cover the edges of the wires comprising them. The circular portion 12 also preferably includes a center cut-out 24 for placing a user's knee or elbow therein.

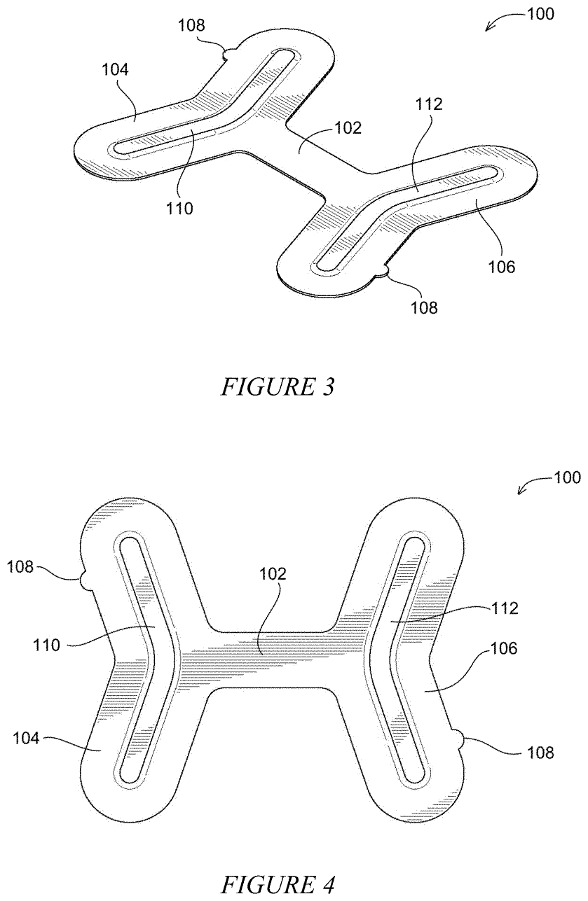

Referring to FIGS. 3 and 4, a second embodiment sports tape for knee and elbow placement (sports tape) 100 includes a central bar portion 102 between a first stabilizer portion 104 and a second stabilizer portion 106. The first stabilizer portion 104 and the second stabilizer portion 106 are preferably arced or chevron-shaped members, with the concave portion of the first stabilizer portion 104 and the second stabilizer portion 106 aimed away from the bar portion 102. Each of the first stabilizer portion 104 and the second stabilizer portion 106 include a tab 108 for exposing an adhesive backing and adhering the sports tape 100 to a user's knee or elbow. Thus, in its preferred embodiment, the second embodiment sports tape 100 is substantially similar to the first embodiment sports tape 10 except for having a central bar portion 102 instead of a central circular portion 12.

Still referring to FIGS. 3 and 4, the first stabilizer portion 104 includes an embedded first stabilizer 110, and the second stabilizer portion 16 includes an embedded second stabilizer 112. The first stabilizer 110 and the second stabilizer 112 preferably follow the arc or chevron-shape of the first stabilizer portion 104 and second stabilizer portion 106, respectively, and in one embodiment are centered thereon as shown in the figures. The first stabilizer 110 and the second stabilizer 112 preferably comprise a resiliently deformable material. In one preferred embodiment, the first stabilizer 110 and the second stabilizer 112 are made from steel spiral corset boning, including safety caps (not shown) at their ends to cover the edges of the wires comprising them. The central bar portion 102 may be of any preferred length or thickness according to preference.

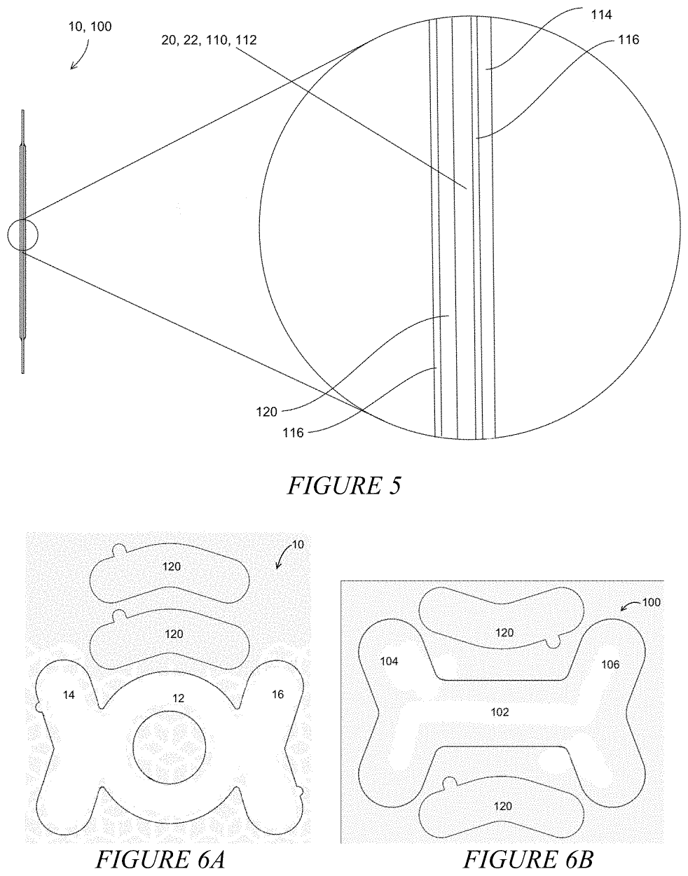

Referring to FIG. 5, a section view of the sports tape 10, 100 (the features discussed in FIG. 5 are the same in both embodiments) is shown in section view with a portion enlarged portion to illustrate its composition. The enlarged portion illustrates a cross section of any of the first embodiment first stabilizer portion 14 or second stabilizer portion 16, and second embodiment first stabilizer portion 104 or second stabilizer portion 106, as these all have the same layers of materials discussed.

The sports tape 10, 100 includes a non-woven tape 114, which is formed of a non-woven cloth, preferably around 30 grams per square meter, which is breathable and easy to tear by hand. The non-woven tape 114 includes an adhesive (not shown) covering the non-woven tape 114 on one side. A transfer tape adhesive 116, which is preferably an approximately 4.5 mil rubber transfer adhesive, is adhered to the adhesive bearing side of the non-woven tape 114. One of the stabilizers (20, 22, 110, or 112) is affixed to the transfer tape adhesive 116 on one of the stabilizer portions (14, 16, 104, or 106). A second non-woven tape layer 120, also having the transfer tape adhesive 116 is located on the opposite side of the stabilizer 12, 22, 110, 112.

The sports tape for knee and elbow placement having been shown and described, it's method of construction will now be discussed.

In order to manufacture the first embodiment sports tape 10 and the second embodiment sports tape 100, the raw materials are first provided, including the non-woven tape 114, the transfer tape adhesive 116, and the stabilizers 20, 22, 110, 112. The stabilizers 20, 22, 110, 112 are preferably all of the same manufacture and shape, preferably being made of sturdy, durable but flexible galvanized metal wires. In one preferred embodiment, the stabilizers 20, 22, 110, 112 are cut to pieces approximately one centimeter wide by approximately 10.8 centimeters long, and having a thickness of preferably approximately one millimeter. Each of the stabilizers 20, 22, 110, 112 are also preferably bent at their lengthwise midpoint at approximately 101 degrees, to have an angle matching the stabilizer portions 14, 16, 104, 106, and have safety caps at their ends to prevent wires from puncturing the non-woven tape 114 or transfer adhesive tape 116.

The manufacturing process begins with rolls of the non-woven tape 114 being sent to a printing facility, and transfer adhesive tape 116 being sent to a converting facility, and with the stabilizers being assembled. An entire roll of non-woven tape 114 is printed with a pattern and/or color, according to preference, and the shapes of the first embodiment sports tape 10 and second embodiment sports tape 100 are printed thereon as shown in FIGS. 6A and 6B. In addition to the first embodiment sports tape 10 or second embodiment sports tape 100, two sides of a second non-woven tape layer 120 are also printed for sandwiching the stabilizers 20, 22, 110, 112 into position on the stabilizer portions 14, 16, 104, 106 also as shown in FIGS. 6A and 6B.

To laminate the non-woven tape roll and transfer adhesive tape roll together, the paper backing on the printed non-woven tape roll is peeled away to expose the adhesive. The transfer tape adhesive roll is unrolled and transferred to the adhesive side of the printed non-woven tape, in the process making the two rolls into one laminate roll. The assembled roll is thus layered with 1) a printed side of the non-woven tape, 2) the original non-woven tape adhesive combined with the transfer tape adhesive, and 3) a wax-paper backing originally part of the transfer tape adhesive. The paper backing that was originally part of the non-woven tape roll is disposed.

After the laminated non-woven tape and transfer adhesive tape roll is created, the assembled roll is cut, by using, for example, a die-cutting machine for high volume production, or a laser or similar cutter for low volume production. Thus, for each of the first embodiment sports tape 10 and the second embodiment sports tape 100, there will be one main design piece, and two second non-woven tape layers 120, as shown in FIGS. 6A and 6B.

The final product is assembled by peeling back the wax-paper backing (formerly of the transfer tape adhesive) from one of the stabilizer portions 14, 16, 104, 106, and removing the wax-paper backing from one of the second non-woven tape layers 120 entirely. A stabilizer 20, 22, 110, 112 is then centrally aligned with the stabilizer portion 14, 16, 104, 106. Once the stabilizer 20, 22, 110, 112 is in position, the wax-paper backing is peeled from a second non-woven tape layer 120 (it having been laminated with the transfer adhesive tape).

The non-adhesive side of the second non-woven tape layer 120 is centered over the stabilizer 20, 22, 110, 112 and the stabilizer portion 14, 16, 104, 106. Preferably the second non-woven tape layer 120 is slightly smaller than each stabilizer portion 14, 16, 104, 106, such that once applied, both the adhesive side of the laminated second non-woven tape layer, and the adhesive side of the non-woven tape and transfer tape adhesive 120 laminate are both exposed. Thereafter the wax-paper backing can be re-installed over the sports tape 10, 100.

The tabs 18, 108 extend outside the main portion of the sports tape 10, 100 to make it easier for a user to split open the product, and expose the stabilizers 20, 22, 110, 112 for future use or recycling. Preferably the non-woven tape material is made from a biodegradable or semi-biodegradable material, along with the transfer adhesive tape, and the stabilizers, and the ink material used for printing on the non-woven tape is also eco-friendly.

To use the sports tape 10, 100, a user first shaves all hair around the knee area and/or elbow area, followed by applying alcohol to the area so that it is clean and dry. The user's knee (or elbow) is matched to the same angle as the side stabilizers, with the stabilizers on either side of the user's knee or elbow. The backing of the sports tape 10, 100 is completely removed. Thereafter, the middle bar is placed just under the user's knee cap (or elbow), or centers the circle on the user's knee cap or elbow. Starting at the middle bar (or circle), a user firmly presses the users fingers and hands across the sports tape 10, 100, working outward toward the stabilizer portions 14, 16, 104, 106. The sports tape 10, 100 is held in place for approximately twenty seconds. To remove the sports tape 10, 100, a user peels the tape with one hand while the other holds the user's skin from rising with it. Materials such as soaps or oil-containing products may help with the removal process.

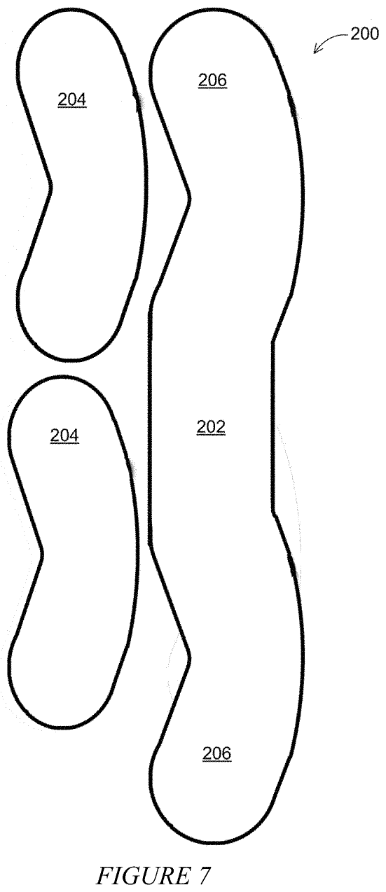

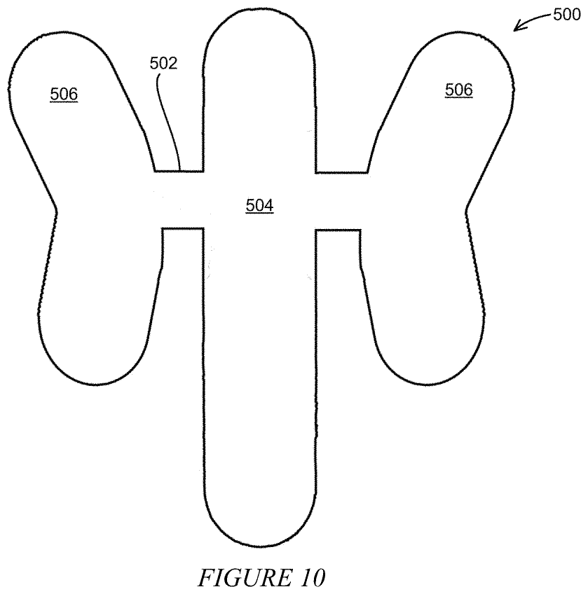

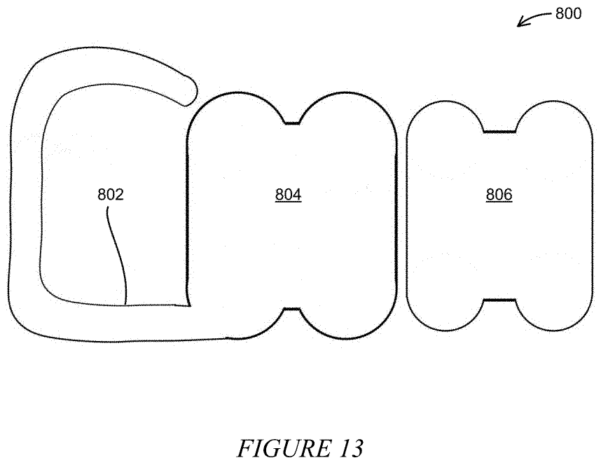

Additional embodiments of the sports tape are shown in FIGS. 7-13, illustrating embodiments adapted for application to a user's ankle (FIGS. 7-9), shoulder (FIG. 10-12), and finger (FIG. 13). Referring to FIG. 7, a first additional embodiment sports tape 200, includes a first woven tape layer 202, and two second woven tape layers 204. The two second woven tape layers 204 are shaped and sized to overlay two extreme ends 206 of the first woven tape layer. In a preferred embodiment, the two second woven tape layers 204 will cover stabilizers (not shown) as in prior embodiments.

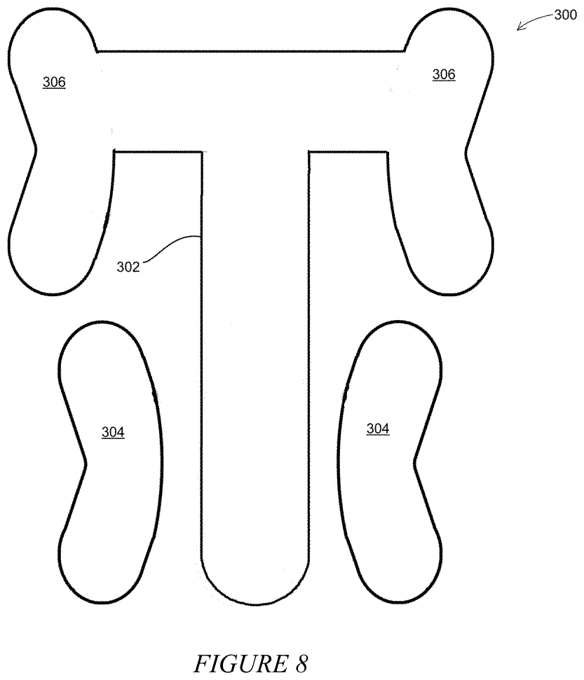

Referring to FIG. 8, a second additional embodiment sports tape 300 for application to a user's ankle is shown. The sports tape 300 includes a first woven tape layer 302, and two second woven tape layers 304. The first woven tape layer 302 is T-shaped, and the two second woven tape layers 304 are shaped and sized to overlay the two ends 306 of the T crossbar. In a preferred embodiment, the two second woven tape layers 304 will cover stabilizers (not shown) as in prior embodiments.

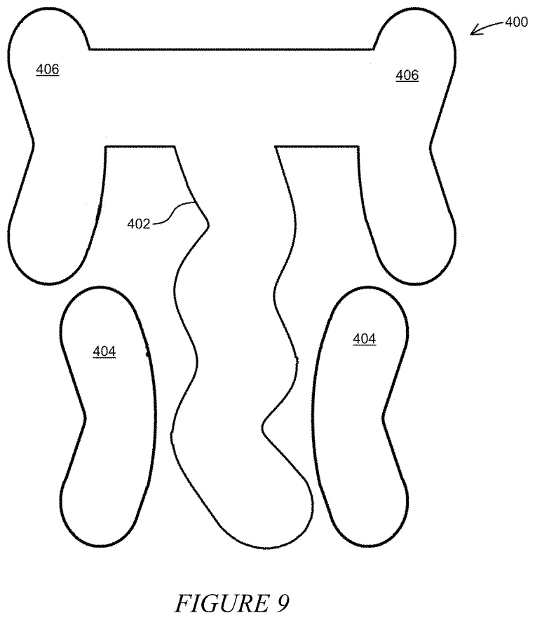

Referring to FIG. 9, a third additional embodiment sports tape 400 for application to a user's ankle is shown. The sports tape 400 includes a first woven tape layer 402, and two second woven tape layers 404. The first woven tape layer 402 is T-shaped with a zig-zag pattern on the leg of the T, and the two second woven tape layers 404 are shaped and sized to overlay the two ends 406 of the T crossbar. In a preferred embodiment, the two second woven tape layers 404 will cover stabilizers (not shown) as in prior embodiments.

Referring to FIG. 10, a fourth additional embodiment sports tape 500, for application to a user's shoulder is shown. The sports tape 500 includes a first woven tape layer 502 having a central leg 504 for application over the center of a user's shoulder, and two side legs 506 for application over the anterior and posterior portions of the user's shoulder. This embodiment has a single connector connecting the central leg 504 to the two side legs 506. Preferably two second woven tape layers and stabilizers (not shown) are affixed to the side legs 506, as in priori embodiments.

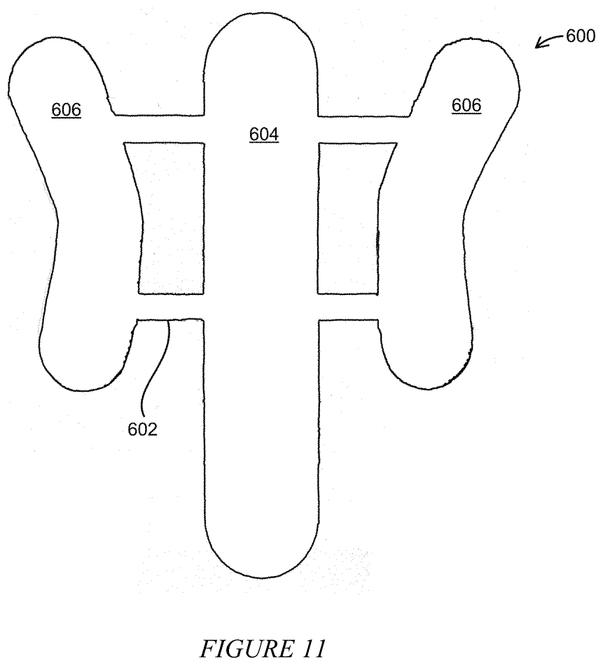

Referring to FIG. 11, a fifth additional embodiment sports tape 600, for application to a user's shoulder is shown. The sports tape 600 includes a first woven tape layer 602 having a central leg 604 for application over the center of a user's shoulder, and two side legs 606 for application over the anterior and posterior portions of the user's shoulder. This embodiment has two connectors connecting the central leg 604 to the two side legs 606. Preferably, two second woven tape layers and stabilizers (not shown) are affixed to the side legs 606, as in priori embodiments.

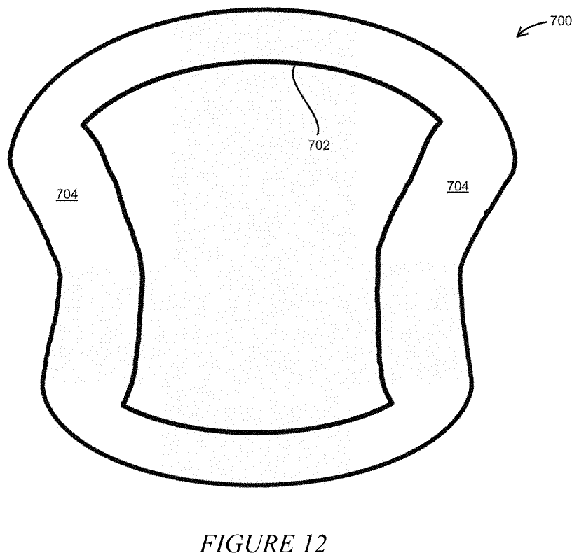

Referring to FIG. 12, a sixth additional embodiment sports tape 700, for application to a user's shoulder is shown. This sports tape 700 includes a first woven tape layer 702, formed as a closed loop, and which includes two side portions 704. The side portions 704 are for installation over the anterior and posterior portions of the user's shoulder. Preferably, two second woven tape layers and stabilizers (not shown) are affixed to the side portions 704, as in prior embodiments.

Referring to FIG. 13, a seventh additional embodiment sports tape 800, for application to a user's finger is shown. This sports tape 800 includes a first woven tape layer 802 having a stabilizing portion 804, that extends over two sides of a user's finger, and a second woven tape layer 806. Preferably the second woven tape layer 806 overlays the stabilizing portion 804 and includes a stabilizer (not shown) installed between them, as shown in previous embodiments.

The foregoing descriptions of embodiments of the present invention have been presented only for purposes of illustration and description. They are not intended to be exhaustive or to limit the present invention to the forms disclosed. Accordingly, many modifications and variations will be apparent to practitioners skilled in the art. Additionally, the above disclosure is not intended to limit the present invention. The scope of the present invention is defined by the appended claims.

What is claimed is:

1. A sports tape for placement at the location of a user's knee or elbow joint, the sports tape comprising:
a first stabilizer portion and a second stabilizer portion, the first stabilizer portion configured for placement on one side of the user's knee or elbow joint, and the second stabilizer configured for placement on an opposing side of the user's knee or elbow joint;
a central portion connecting the first stabilizer portion and the second stabilizer portion, the central portion configured to wrap around the user's knee or elbow joint;
a first stabilizer embedded in the first stabilizer portion, and a second stabilizer embedded in the second stabilizer portion, the first stabilizer and the second stabilizer each centered on the first stabilizer portion and the second stabilizer portion, respectively; and
wherein the first stabilizer and the second stabilizer are disposed inside the sports tape, sandwiched between sports tape layers.

2. The sports tape of claim 1 wherein the sports tape comprises a non-woven tape with adhesive.

3. The sports tape of claim 1 wherein the stabilizers comprise steel corset boning material.

4. The sports tape of claim 1 further comprising a transfer tape adhesive for adhering the sports tape to the user's knee or elbow.

5. The sports tape of claim 1 wherein the first stabilizer portion and the second stabilizer portion are elongated members with a middle bend in the direction of the user's knee or elbow joint.

6. The sports tape of claim 1 wherein the central portion is a bar between the first stabilizer portion and the second stabilizer portion.

7. The sports tape of claim 1 wherein the central portion is a planar circle, with the first stabilizer portion and the second stabilizer portion located on opposing sides of the circle.

8. The sports tape of claim 1 further comprising a non-woven tape layer covering the first stabilizer and a non-woven tape layer covering the second stabilizer.

9. A method of manufacturing a sports tape for placement at the location of a user's knee or elbow joint, the method comprising the steps of:
obtaining a sheet of non-woven tape having an adhesive side and a non-adhesive side;
obtaining a sheet of transfer tape adhesive;
obtaining a length of corset boning;
printing a design corresponding to the sports tape outline on the non-adhesive side of the non-woven tape;
laminating the transfer tape adhesive to the adhesive side of the non-woven tape;
cutting the non-woven tape, after laminating, into a plurality of sports tape shapes for placement at the location of a user's knee or elbow joint, and a plurality of second non-woven tape layers;
placing the boning on the sports tape shape, on the transfer tape adhesive;
placing one of the second non-woven tape layers over the boning on the printed side of the second non-woven tape layer, thereby having uniform transfer tape adhesive coverage over the boning; and
placing the sports tape proximal the user's knee or elbow joint.

10. The method of claim 9 further comprising the step of cutting the non-woven tape, after laminating, into a shape having a bar configured for placement at the user's knee or elbow joint.

11. The method of claim 9 further comprising the step of cutting the non-woven tape, after laminating, into a shape having a planar circle configured for placement at the user's knee or elbow joint.

12. The method of claim 9, further comprising the step of cutting the non-woven tape, after laminating, to form two stabilizer portions on either side of a central portion.

13. The method of claim 12, wherein the stabilizer portions are angled complimentary to the user's knee or elbow joint.

14. The method of claim 12, wherein the lengths of corset boning are located on the stabilizer portions.

15. The method of claim 14, wherein the lengths of corset boning are centered on the stabilizer portions.

16. The method of claim 9 further comprising the step of placing a wax paper layer on the transfer tape adhesive opposite the non-woven tape.

17. A method of using an improved sports tape for placement at the location of a user's knee, elbow, shoulder, or finger joint, the method comprising the steps of:
shaving the area around the user's knee, elbow, shoulder, or finger joint;
applying rubbing alcohol around the area around the user's knee, elbow, shoulder, or finger joint;
matching the user's knee, elbow, shoulder, or finger joint to a pair of side stabilizers on the sports tape;
peeling a backing off the sports tape;
while centering the sports tape, placing the middle of the sports tape adjacent the middle of the user's knee, elbow, shoulder, or finger joint;
working the sports tape against the user's knee, elbow, shoulder, or finger from the center outward;
pressing and holding the sports tape against the user's knee, elbow, shoulder, or finger joint for approximately twenty seconds; and
engaging in athletic activity while wearing the sports tape.
